(19) United States
(12) Patent Application Publication
(10) Pub. No.: US 2021/0074400 A1
(43) Pub. Date: Mar. 11, 2021

(54) SYSTEM AND METHOD FOR PERFORMANCE-BASED MANAGEMENT OF THERAPEUTIC PRODUCTS

(71) Applicant: Seth Feuerstein, New Haven, CT (US)

(72) Inventor: Seth Feuerstein, New Haven, CT (US)

(21) Appl. No.: 17/012,688

(22) Filed: Sep. 4, 2020

Related U.S. Application Data
(60) Provisional application No. 62/896,162, filed on Sep. 5, 2019.

(51) Int. Cl. G16H 20/00; G16H 40/63; G16H 40/67; G06F 3/0481; G16H 10/60; G06Q 30/02

(52) U.S. Cl. CPC G16H 20/00; G16H 40/63; G16H 40/67; G06Q 30/0203; G16H 10/60; G06Q 30/0283; G06F 3/0481

(57) ABSTRACT

A system and method for treatment efficacy-based evaluation of therapeutic products, and management of distribution of, and efficacy-based determination of payments for, such therapeutic products. A computerized therapeutics management system is provided that monitors a broad range of activities within a communications/data network to obtain relevant data, allow payors to define what types of therapeutic benefits/efficacy they value and are willing to pay for by selection of corresponding efficacy metrics, e.g., via a corresponding graphical user interface, using data from disparate sources to evaluate efficacy of therapeutic products using the payor-selected efficacy metrics, and then providing for payments to be established, and/or controlling payors' or other's systems to make payments, as a function of efficacy as measured by the payor-selected metrics. The system thereby improves the treatment of care for patients, an reduces overall health system costs.

### CROSS-REFERENCE TO RELATED APPLICATION

This application claims the benefit of priority, under 35 U.S.C. § 119(e), of U.S. Provisional Patent Application No. 62/896,162, filed Sep. 5, 2019, the entire disclosure of which is hereby incorporated herein by reference.

### FIELD OF THE INVENTION

The present invention relates generally to treatment of patients with medically therapeutic products intended to provide a medically beneficial, or “therapeutic”, benefit to a patient in the nature of treatment or healing of a disease, and more particularly to systems and methods for treatment efficacy-based evaluation of digital therapeutic-type products.

### DISCUSSION OF RELATED ART

In the field of medicine and healthcare, there are many beneficial or therapeutic medical devices, pharmaceutical products, and the like. Many such products are FDA-regulated medical products that are available only as prescribed by a licensed healthcare provider.

As is well-known, health insurance plans are commercially available that provide for payments, often at negotiated discounted rate, for such therapeutic products that are prescribed to members of the various health insurance plans.

Further, as is well-known, payments for these therapeutic products may vary over time, and across insurance plans. Some products may be added to a list of formulary products, as those that are preferred as being sufficiently effective and cost-efficient, and thus offering the greatest value. By way of example, a committee of independent, actively practicing physicians and pharmacists may maintain a formulary, which may chance over time. Extensive clinical studies or experience may be involved in making decisions to update the formulary. It is well known that payors have struggled to match payments to actual value delivered by products they pay for, how to define that value and measure the value.

Relatively recently, it has been observed that the healthcare system may benefit from the potential advantages of medical treatment software applications, e.g., to provide a beneficial or medically therapeutic benefit to a patient. The promise of “digital health” has been discussed in popular and industry media for several years.

Digital software applications (i.e., “apps”) enabling improvements in health (“digital therapeutics”) are emerging. For example, there are now apps that purport to treat a patient, and provide a therapeutic effect, with respect to depression, addiction, diabetes, and other medical conditions. Some digital therapeutics might be “prescribed” to improve medication compliance or that seek to lower the cost of care.

Some of these apps are promoted with promises explaining they achieve certain values such as improvement in health, lower costs or improvement in health-related metrics. However, many do not deliver the value promised. For example, an app may purport to improve depression, but this may only be the case in a small portion of users, or it may only partially alleviate the patient's suffering.

The FDA has started to approve “software as a medical device.” For example, the commercially-available reSET and reSET-O digital therapeutic products (DTPs) of Pear Therapeutics, Inc. are smartphone-/table-type software applications that have been “approved” (the term “approved” being used broadly here) by the Food and Drug Administration (FDA). By way of example, the reSET-O DTP is available via prescription only for use to provide a therapeutic effect to patients of opioid use disorder by providing cognitive behavioral therapy (CBT) via these DTPs. By way of further example, the commercially-available AKL-T01 DTP of Akili Interactive Labs is a smartphone/tablet-type software application that is FDA-approved for use to provide a therapeutic effect to ADHD patients, by delivering a video game-type experience designed as “digital medicine.”

Digital therapeutic apps are being created quickly, and the medical/healthcare/insurance system is not well equipped to identify which ones to use, to understand their efficacy, and to evaluate how to price them and offer them seamlessly to consumers, or help providers know which ones are covered. Despite announcements that such entities plan to address these problems, no solutions currently exist as has been done traditionally for drugs. One of the key reasons for this is the rapid pace of development, uncertainty about value to the patients and to the healthcare system, and a lack of systems to manage these products. Legacy methods such as the “formulary” are slow to react to new data in the face of products that bring new data, faster data and new types of data. Even if one were to create a formulary for digital applications, traditional evaluation methodologies would not harness the unique value that digital products might bring to healthcare. There has not been clarity from the insurance/payor community about what they will pay for, how they will pay for it, appropriate payment magnitudes, etc.

Even for established therapeutic products other than software, there is variation as to payment amounts across payors, and payment amounts are typically pre-negotiated on a per-insurance plan basis. Payments are typically made based on claims, or occurrences—for example, a fixed payment may be made for 30 doses of a particular medication, or for a particular medical device. Accordingly, for example, medication may be paid for without regard to whether the patient actually takes the medication, or without regard to whether the patient takes the medication, but the patient's condition does not improve, or whether the patient takes the medication and the patient's condition does improve, although only the latter is most beneficial to the patient and to the healthcare system in terms of providing effective patient care at lower overall cost. There is no mechanism that allows a particular payor to establish efficacy-based criteria of the payor's choosing, or evaluation of efficacy/efficacy (collectively herein, “efficacy”) of a therapeutic product based on that criteria, or determining payment amounts based on efficacy on a particular patient or group of patients.

What is needed is system and method for treatment efficacy-based evaluation of digital and other therapeutic products, and management of distribution of, and payments for, such therapeutic products. The present invention fulfills these needs, among others.

### SUMMARY

The present invention provides a system and method for treatment efficacy-based evaluation of therapeutic products, and management of distribution of, and efficacy-based determination of payments for, such therapeutic products. A computerized therapeutics management system is provided that monitors a broad range of activities within a communications/data network to obtain relevant data, allow payors to define what types of therapeutic benefits/efficacy they value and are willing to pay for by selection of corresponding efficacy metrics, e.g., via a corresponding graphical user interface, using data from disparate sources to evaluate efficacy of therapeutic products using the payor-selected efficacy metrics, and then providing for payments to be established, and/or controlling payors' or other's systems to make payments, as a function of efficacy as measured by the payor-selected metrics.

In certain embodiments, the system allows for payments for a particular therapeutic product for a particular patient to be determined and paid based on the efficacy/outcome for a group of patients that may or may not include the particular patient, as a result of that particular therapeutic product. Accordingly, value/pricing/payments for a therapeutic product is based on the actual observed therapeutic effect/benefit.

In other embodiments, the system allows for payments for a particular therapeutic product for a particular patient to be determined and paid based on the efficacy/outcome for that particular patient as a result of that particular therapeutic product. Accordingly, value/pricing/payments for the same therapeutic product may vary across multiple different patients, based on the actual observed therapeutic effect/benefit for each respective patient. Accordingly, the value/pricing payments for a particular therapeutic product for a particular patient is determined according to the actual observed benefit/value to that particular patient. The system thereby improves the treatment of care for patients, and reduces overall health system costs.

### BRIEF DESCRIPTION OF THE FIGURES

An understanding of the following description will be facilitated by reference to the attached drawings, in which:

FIG. 1 is a system diagram showing an exemplary network computing environment in which the present invention may be employed;

FIG. 2 is a schematic diagram of an exemplary computerized system in accordance with an exemplary embodiment of the present invention; and

FIG. 3 is a block diagram illustrating data flows within the network computing environment of FIG. 1.

### DETAILED DESCRIPTION

The present invention relates to treatment of human patients with therapeutic products, and computerized systems and methods for efficacy-based evaluation of therapeutic products, and management of distribution of, and payments for, such therapeutic products on computerized computing devices within a communications/data network environment.

Although the present invention is applicable to a broad range of therapeutic products, the present invention is described herein with reference to digital therapeutic products in particular, for illustrative purposes only. More specifically, the present invention provides a Therapeutics Management (TM) system and method for efficacy-based evaluation of digital (or other) therapeutic products, such as software, that provides a medically therapeutic beneficial effect to a patient by way of operation of/interaction with the software/therapeutic product. Accordingly, distribution/approval/payments for therapeutic products may be managed/assessed and/or determined according to the efficacy of a therapeutic product, leading to greater distribution/approval/payments for more effective (higher-value) products, and lesser distribution/approval/payments or less effective (lower-value) products, which serves to align the interests and incentives of therapeutic product providers and medical bill/insurance payors (“payors”).

According to certain embodiments of the present invention, there is provided a method of evaluating digital therapeutic products (software applications) using various value-capturing metrics that were previously unmeasurable by payors. Further, these metrics may be selected by the payor, e.g., via a graphical user interface displayable to an agent of the payor via a display of a computerized device. For example, one payor may value metrics that demonstrate patient engagement/compliance, another payor may value metrics that demonstrate that a patient feels better, or sleeps better, or is in an overall happier state, and another payor may value a decreased frequency of healthcare visits, or a reported improvement of the patient's physical, mental or emotional state. Accordingly, the present invention allows each payor to define or choose (e.g., via selections made via a graphical user interface) measures of perceived value, and to ultimately pay for the therapeutic according to the therapeutic's value, measured as that particular payor perceives it. Ultimately, the system of the present invention manages distribution and approval of therapeutic products such that patients can have better medical outcomes/results, and the cost of medical/health care can be lowered.

### Computing Environment

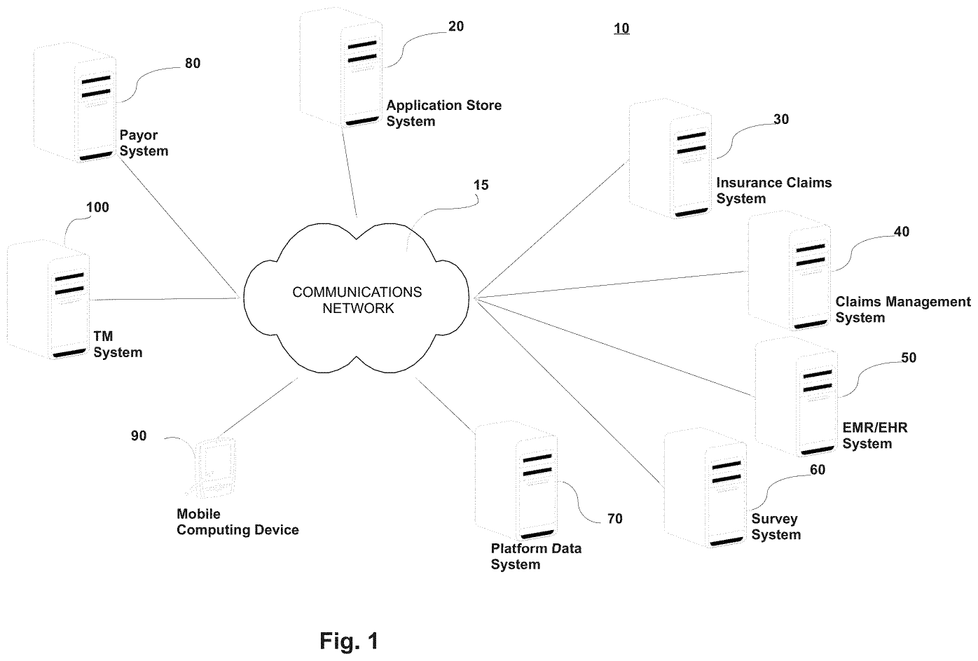

An exemplary embodiment of the present invention is discussed below in greater detail for illustrative purposes. FIG. 1 is a system diagram showing an exemplary network computing environment 10 in which the present invention may be employed. As shown in FIG. 1, the exemplary network environment 10 includes a conventional Application Store System 20, such as the Apple App Store system or the Google Play system that provides an online interface for obtaining/purchasing software applications/apps for Apple iOS and Google Android devices, respectively, which may include conventional software and web server hardware. As further illustrated by FIG. 1, the exemplary network computing environment 10 further includes a Patient Computing Device 90, while may be a smartphone or other mobile computing device, or a desktop, notebook, laptop or other personal computing device, each of which may include conventional hardware and software for communicating via a communications network 15, such as the Internet. The exemplary network computing environment 10 also includes other conventional computerized systems, such as an Insurance Claims System 30 and a Claims Management System 40 (such as those used by medical insurance companies and/or in the medical insurance industry), an EMR/EHR System 50 (such as those commercially available from EPIC or Cerner or ALLSCRIPTS), a Survey System 60 (such as SurveyMonkey), and a digital therapeutic product's Platform Data System 70, such as the server/system that supports the commercially-available PEAR DTP app, or the AKILI DTP app, or any other DTP app's related system, and a Payor System 80 (such as those used by companies such as United Healthcare, Magellan and AETNA), as will be appreciated by those skilled in the art. These systems may include existing or otherwise generally conventional systems including conventional software and web server or other hardware and software for communicating via the communications network 15. Consistent with the present invention, these systems may be configured, in conventional fashion, to communicate/transfer data via the communications network 15 with the TM System 100 in accordance with and for the purposes of the present invention, as discussed in greater detail below.

The exemplary computing environment 10 also includes a Patient Computing Device 90. The Patient Computing Device 90 may include conventional computing hardware storing and executing conventional software enabling operation of a general-purpose computing system. As such, Patient Computing Device 90 may include hardware and software similar to that of the TM System 100, but need not include the special-purpose software described with reference to the TM System 100, and the communications software 130 may alternatively include conventional web browser software, and the operating system software 120 may include iOS, Android, Windows, or Linux software. Notably, a DTP app is stored or storable in the memory of the Patient Computing Device 90 to configure the Patient Computing Device 90 to provide the therapeutic or other beneficial effects to the patient by way of the patient's interaction with the DTP app via the Patient Computing Device 90. In one embodiment, the digital therapeutic app does not need to be configured in any particular way to deliver the benefits of the present invention. Preferably, the digital therapeutic is configured to communicate data that may be used to measure efficacy/efficacy and/or value to the TM System 100 and this may be done using a conventional data communications mechanism, e.g., via an API. In certain embodiments, the digital therapeutic app may be configured in accordance with the present invention to gather particular data relating to the user, the app, and/or the patient's/user's interaction with the app, to provide relevant data to the TM System 100 for measuring efficacy/efficacy and value, depending upon the information desired to be gathered by the TM System 100.

In accordance with the present invention, the network computing environment 15 further includes a Therapeutics Management (TM) System 100. In this exemplary embodiment, the TM System 100 is operatively connected to the Patient Computing Device 90, and to the other systems shown in FIG. 1, for data communication via the communications network 15. Hardware and software for enabling communication of data by such devices via such communications networks are well known in the art and beyond the scope of the present invention, and thus are not discussed in detail herein.

### Therapeutics Management System

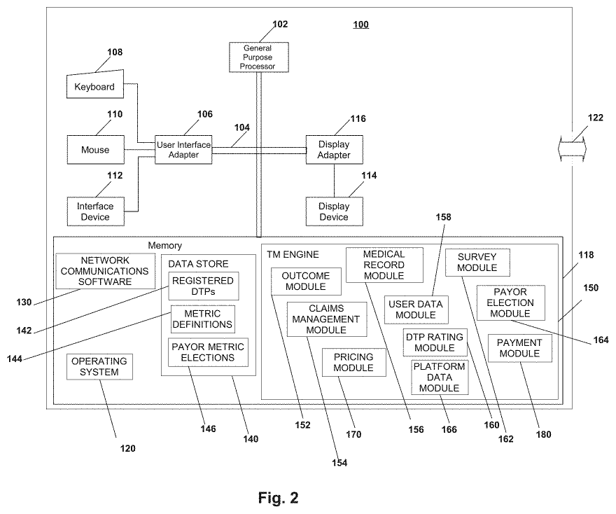

FIG. 2 is a block diagram showing an exemplary Therapeutics Management (TM) System 100 in accordance with an exemplary embodiment of the present invention. The TM System 100 includes conventional computing hardware storing and executing both conventional software enabling operation of a general-purpose computing system, such as operating system software 120, network communications software 130. Additionally, TM System 100 includes specially-configured computer software for configuring the general-purpose hardware as a special-purpose computer system for carrying out at least one method in accordance with the present invention. By way of example, the communications software 130 may include conventional web server software, and the operating system software 120 may include iOS, Android, Windows, or Linux software.

Accordingly, the exemplary TM System 100 of FIG. 2 includes a general-purpose processor, such as a microprocessor (CPU), 102 and a bus 104 employed to connect and enable communication between the processor 102 and the components of the presentation system in accordance with known techniques. The exemplary presentation system 100 includes a user interface adapter 106, which connects the processor 102 via the bus 104 to one or more interface devices, such as a keyboard 108, mouse 110, and/or other interface devices 112, which can be any user interface device, such as a touch sensitive screen, digitized entry pad, etc. The bus 104 also connects a display device 114, such as an LCD screen or monitor, to the processor 102 via a display adapter 116. The bus 104 also connects the processor 102 to memory 118, which can include a hard drive, diskette drive, tape drive, etc.

The TM System 100 may communicate with other computers or networks of computers, for example via a communications channel, network card or modem 122. The TM system 100 may be associated with such other computers in a local area network (LAN) or a wide area network (WAN), and may operate as a server in a client/server arrangement with another computer, etc. Such configurations, as well as the appropriate communications hardware and software, are known in the art.

As shown in FIG. 2, the TM System 100 includes computer-readable, processor-executable instructions stored in the memory 118 for carrying out the methods described herein. Further, the memory 118 stores certain data, e.g. in one or more databases or other data stores 140. The data/data stores and modules enabled by the stored instructions are shown logically in FIG. 2 for illustrative purposes, without regard to any particular embodiment in one or more hardware or software components.

Further, as will be noted from FIG. 2, the TM System 100 includes, in accordance with the present invention, a TM Engine 150, shown schematically as stored in the memory 118, which includes a number of additional modules providing functionality in accordance with the present invention, as discussed in greater detail below. These modules may be implemented primarily by specially-configured software including microprocessor-executable instructions stored in the memory 118 of the TM System 100. Optionally, other software may be stored in the memory 118 and and/or other data may be stored in the data store 140 or memory 118.

Further, in accordance with the present invention, one embodiment of the TM System 100 includes a list of registered digital therapeutic products (DTPs) 142. DTPs are added to the list of registered DTPs by a system operator, or otherwise in response to approval by the FDA, after which they would automatically be listed on an FDA database. By way of example, this may be achieved using a suitable graphical user interface that is caused to be displayed by the TM System 100, e.g., on its display device, or on the display device of a remotely-located computing system in communication with the TM System 100 via the communications network 15. For example, a list including a particular depression-treating DTP and a particular smoking-cessation DTP, and identifying particular apps downloadable from commercial App Stores, may be included in the list of registered DTPs 142, for example, as a result of application to and approval of, or otherwise making those DTPs available as part of the use of the TM System.

Further, the exemplary TM System 100 includes DTP Metric Definitions 144 that can be selected by payors to evaluate DTPs stored in its data store 140. Examples of metrics include reduction in total healthcare spending, improvement in a clinical measure (such as blood pressure, blood sugar measurements, improvement in PHQ-9, weight change, hospital readmissions, etc.) By way of further example, a Metric 1 definition may provide for improvement on a clinical scale included in the app and a Metric 2 definition may provide for comparison of the cost of care and a Metric 3 definition may be the rate of hospital readmission of the users and a Metric 4 definition may be consumer satisfaction information and a Metric 5 definition may be the patient/'s actual weight change or blood sugar level or score on a pain scale or sleep quality. The metrics provide different ways in which to evaluate, quantify, track or otherwise capture degrees of value, and thus they can be used to differentiate among DTPs, and may be chosen by payors to align the payor's motivations with efficacy of the DTPs. Any suitable metric definitions may be used.

The Metric Definitions 144 may also include information provided by a software developer/publisher of a DTP indicating the metrics the DTP/DTP developer reports will improve, for review and comparison to actual outcomes when in use in the real world. For example, a DTP developer/publisher may claim that its DTP reduces relapse of addiction. This can be entered by the DTP developer/publisher as a measure of value, and then can be compared to actual outcomes of relapse as determined by claims for readmission or data entered by the member or their clinicians in an EMR via a corresponding Metric Definition.

Further, the exemplary TM System 100 includes DTP Payor Metric Elections 146, indicating one or more metrics, from the Metric Definitions 144, that have been elected for use in association with each payor, to the extent such elections have been made and stored in the data store 140 of the TM System 100. By way of example, this may be achieved using a suitable graphical user interface that is caused to be displayed by the TM System 100, e.g., on its display device, or on the display device of a remotely-located computing system in communication with the TM System 100 via the communications network 15, and used by an agent of the payor. By way of example, the Payor Metric Elections 146 may indicate that Payor 1 has selected Metric 1 as set forth in the Metric Definitions 144, and the Payor Metric Elections 146 may indicate that Payor 2 has selected Metric 2 as set forth in the Metric Definitions 144. Accordingly, the TM System 100 will use each payor's elected metric(s), as recorded by the TM System 100 in the Payor Metric Elections 146, in evaluation of the DTP for distribution/approval/pricing and payment purposes for each respective payor, as discussed further below.

The TM System 100 may also provide, and cause to be displayed at each of their respective computing devices, graphical user interface (GUI) dashboards to allow patients, providers and payors to each see relevant data for each of them, and to help them choose a suitable or best DTP app for a patient, e.g., by the Interface Module's 182 communication of data via the Communications Network 15 to present corresponding web pages for viewing via patients' and others' computing devices. For instance, there may be multiple DTPs for a single diagnosis such as insomnia. The TM System 100 compares the DTPs showing data from academic, clinical, population and patient usage in general and in different subpopulations such as based on age or gender or consumer feedback. For example, while there may have only been a few published, controlled studies of a DTP, the system may compare outcome data from highly specific patient cohorts and if a patient is female, has diabetes, is over 65 years of age and is taking certain medications the system may examine outcome data across all such patients in the overall system and recommend a specific DTP for insomnia that performs especially well in that type of patient, based on an analysis of the outcome data.

Further, the GUI dashboards may be configured to link with and/or display data from external EMR, PHR or e-prescribing systems. For example, there may be health record data pertaining to a specific individual contained in one of these systems in relation to another illness, and the system may determine that a particular DTP is not a good fit, or is a particularly good fit, for that individual based on such health record data. Such data may also, for example, show that the specific individual has been prescribed a certain medication that has side effects, and the system may determine that a particular DTP is now available to be offered with no side effects, such that the specific individual could stop taking that medication. The relevant stakeholders, such as payor, clinician or patient can be notified of this change by the system. For instance, a symptom or diagnosis for a patient may be captured in EMR/PHR data of an external system. A clinician may want to see all of the available DTPs as well as the data associated with their efficacy, including subsets of data related to that particular patient's age, sex, other confounding diagnoses, and/or according to certain patient or clinician preferences (for instance, a patient may prefer a DTP that has less data on its effectiveness but that has a lower cost). By way of alternative example, a clinician may prefer a DTP that has been studied in a certain age group or has data available from that age group. Accordingly, the GUI dashboards may allow for searching, filtering, etc. to allow for display of a list of suitable DTPs.

### Payor-Specific Value Determination

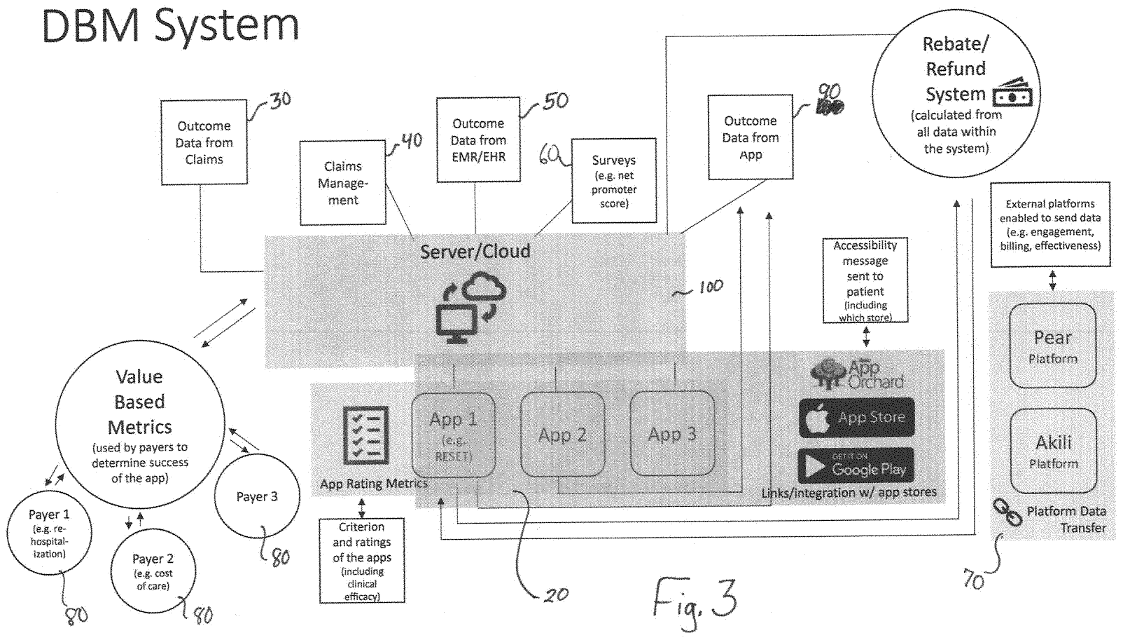

FIG. 3 is a block diagram illustrating selected data flows and interrelationships involving the TM System 100 and the network computing environment of FIGS. 1 and 2. Referring now to FIGS. 2 and 3, it will be appreciated that the TM Engine 150 of the TM System 100 is responsible for communication with external systems via the communications network 15 and for performing many of the functions in accordance with the present invention.

The Application Store System 20 stores an inventory of distributable software products, some of which are DTPs, i.e., software applications, such as smartphone “apps,” designed to provide a beneficial/therapeutic benefit to a user of the app (sometimes referred to herein as the patient). Some of these may be approved for use and distribution as part of the TM System 100. Such approval may be obtained by working with a federal agency such as the FDA. Once approved, the corresponding app may be added through a dashboard available to the payor to the list of registered DTPs 142 stored in the data store 140 of the TM System 100. The list of approved DTPs can be augmented by action of the payor or digital therapeutic product vendors. This may be done via a GUI provided by the TM System 100 and made accessible to the payor.

As shown in FIG. 2, the TM Engine 150 of the TM System 100 includes a Payor Election Module 164. This module enables communication of the TM System 100, via the communications network 15, with the Payor System 80, such that a user of the Payor System is presented with web-pages or other suitable electronic communications effectively providing the user with a user interface such that the user can identify DTPs that it wishes to authorize for payment by the payor. By way of example, individual payors may enter their elections via a web-based dashboard graphical user interface displayed on the Payor System 80 (which may include a payor's PC or other computing device (not shown) connected to the TM System 100 via the communications network). For example, the user may be presented with a display of the list of registered DTPs 142 as defined in the Data Store 140. The Payor may browse the list and be presented with a predefined list of metric definitions (retrieved from the Metric Definitions 144 stored in the Data Store 140) that may be used to evaluate efficacy for the associated DTP. Notably, the Payor may be presented with metric-based efficacy data associated with the various DTPs (as stored in association with the DTPs in the Registered DTP data 142). This allows Payors to browse competing DTPs (e.g., for a particular indication) and select a DTP for which it wishes to pay based on the reported/stored metric data. For example, a DTP may include metric-based efficacy data indicating that it provides for high user engagement, or is successful in resolving a condition as evidence by a decreased number of doctor's visits following use of the DTP, or that has demonstrated efficacy supported by a lesser or greater degree of clinical study data, and the Payor may choose a preferred DTP, or payments structures for the various DTPs, based on the reported past efficacy data. The presentation of this list (at the Payor System 80) is managed at least in part by the Payor Election Module 164 of the TM Engine 150.

Further, the user may enter the payor's election of one or more of the metrics for use by the TM System 100 to evaluate future efficacy, or patient-specific efficacy. Alternatively, the Payor Election Module 164 may cause to be displayed at the Payor System 80 a graphical user interface permitting the user to define its own metric to be used. Any user-defined metric is stored by the system in the Metric Definitions 144 of the Data Store 140. Any selection of pre-defined or user-defined metrics is stored in the Payor Metric Elections 146 of the Data Store 140. These elections will subsequently be used by the Pricing Module 170 of the TM Engine 150 to determine, on an efficacy/efficacy basis, whether payments should be made, the amount of payments that should be made, and the timing of payments to be made in accordance with the TM System's 100 evaluation of the DTP's efficacy/efficacy, using the elected metric(s). For instance, a payor may prefer a DTP that promises to lower a patient's blood sugar level and can pay $X for each unit of blood sugar lessening (or lessening as compared to expected increases). In a patient with anxiety, the payor may want to pay part or all of the fee based on the realized improvement in a patient's anxiety level, or productivity at work, or according to a rating of efficacy entered by their clinician, or that is found in the EMR or in the future claims data. Additionally, a DTP may be designed to treat a condition such as heart disease, and may be configured to receive bonus payments if there are fewer hospitalizations or a lower total cost of care, according to metrics. The metrics may be defined and pre-populated within the system, or may be defined by the vendor. The system permits each payor to select from a menu of available metrics which metric or metrics the payor wishes to use to evaluate efficacy of the digital therapeutic product or to negotiate this directly with the vendor and enter it manually. By allowing each payor to individually select metrics to be used for evaluation purposes for the payor, each payor is thereby able to pay for different amounts according to efficacy/efficacy as measure by value metrics based on each payor's own priorities.

The TM System 100 can receive inputs from a list of common value metrics (such as cost savings) or intake custom metrics (such as a formula a payor is interested in that might relate to other information they can link or that is already in the system), or use default metrics, such as an impact of a particular DTP app on claims costs and population health independent of the individual member. Depending upon the nature of the metric, this may require gathering of data from external systems to calculate or otherwise determine the metric or efficacy of the therapeutic product according to the evaluation scheme defined by the metric.

Metric-Based Interface with External Systems

In addition, the TM System 100 may communicate/interface with external system to accumulate individual, population and academic data to confirm and provide metric-based efficacy data for the digital therapeutic products. It may do so by interfacing with other external data systems, as will be appreciated from FIG. 1. For example, the TM System 100 may interface with other external conventional computing systems, via a communications network, to gather additional data that can be used to advantage in accordance with the present invention. For example, the TM System 100 may communicate with an Insurance Claims System 30 that receives from a vendor a datafeed including invoice data, and then processes the invoice data, as shown in FIGS. 1 and 3. By way of example, the Insurance Claims System 30 has claims data for patients, and the insurance claims data for a particular patient provides metric-relevant data that can be used to determine efficacy according to the metric. For example, if the metric involves decreasing the number of doctor's visits during or after use of the DTP, then doctor's visit data from the claims data for the patient that is stored in the Insurance Claims System 30 can be communicated to the TM System 100 and used to evaluate efficacy of use of the DTP app for that particular patient (or for a group/population of patients), using a corresponding metric. By way of further example, if the metric involves reducing the total cost of care, the corresponding data can be retrieved from the claims data of the Insurance Claims System 30 and be communicated to the TM System 100 for evaluating efficacy of the associated DTP using the appropriate metrics.

The TM System 100 may interface with other external data systems via the communications network 15, as will be appreciated from FIG. 1. By way of further example, the TM System 100 may gather additional data for use in accordance with the present invention from a Claims Management System 40 (such as whether a patient has been to the hospital in the year after the DTP use was initiated), as shown in FIGS. 1 and 3. As well-known in the art, such systems store patient-specific medical claims data and are the basis on which services such as doctors visits and hospital stays are paid for. Therefore, the data in these systems can be used to advantage in accordance with the present invention. For example, the TM System 100 may communicate with the Claims Management System 40 to obtain medical claims management data gleaned from insurance claims. This may be done by receiving member record data (for the patient) from the Claims Management System 40, or receiving claims record data at the TM System 100 and processing that data to determine outcomes.

In somewhat analogous fashion, the TM System 100 may also gather additional data for use in accordance with the present invention from an Electronic Medical Records (EMR) or Electronic Health Records (EHR) System 50 (such as EPIC or Cerner), as shown in FIGS. 1 and 3. As well-known in the art, such EHR/EMR systems store patient-specific records of medical and health information, and collectively provides population-specific outcome data, that can be used to advantage in accordance with the present invention to pay for actual impact on the patient's health status change (and if there is no improvement, might support no payment by the payor).

For example, the TM System 100 may communicate with the EMR/EHR System 50 to gather additional data about patient outcomes for a specific DTP broadly, or for a specific patient's health improvement following use of the DTP. Accordingly, actual patient health data (such as blood pressure, weight, blood sugar level data, etc.) can be used to evaluate the DTP's efficacy, and be used to determine the payor's payment.

The system may also track and provide for DTP payments for total population health improvement. For example, it may be the case that hospital readmission rates are difficult to determine for certain populations such as those with heart failure. A DTP may advertise that they reduce readmissions if their DTP is used by patients. The TM System 100 can compare the claims data from those who use the DTP to those that do not and pay for the change between these groups. It may be that the rate of rehospitalization in one group is 50% but in the other groups is 48%. This seemingly small percentage can be significant in overall spending and population health, but may not be measurable on a single patient level. This allows for evaluation of the benefits of the DTP on a particular patient (which may be used to define success of the DTP app for a particular patient, and to be the basis for determining a success-based payment, which can vary based on the degree of success, to be paid by the payor) or for a change in the outcomes of a population as reflected in corresponding data.

In addition to gathering patient and/or population health data from Insurance Claims Systems 30, Claims Management Systems 40 and EMR/HER Systems 50, the TM System 100 may gather additional data for use in accordance with the present invention from a Survey System 60 (such as questionnaires in an app, or third party surveys, for instance from companies like Press Ganey), as shown in FIGS. 1 and 3. As well-known in the art, such systems may be used to present electronic or other questionnaires to people, such as medical professionals, healthcare providers, and patients, so that their responses may be received and stored as appropriate data by the Survey System 60. For example, the TM System 100 may communicate with the Survey System 60 to obtain quality or satisfaction data such as those needed for HEDIS or STARS scores data that can be used to advantage in accordance with the present invention. For example, the TM System 100 may communicate with the Survey System 60 to obtain net promoter score or provision of access to treatment data gleaned from survey responses. For example, the TM System 100 may be configured to communicate with the Survey System 60 to gather subjective customer experience, which may be used, for example, as part of a metric for evaluating a DTP and determining pricing/payments according. For example, the survey system may allow patients to answer surveys about what is important to them to help them select DTP apps. For example, a certain drug might cause weight gain and ease pain in 50% of users. Another drug may ease pain in 40% of users but not cause weight gain. Some users who strongly do not want to gain weight can receive a questionnaire to help guide their “personal precision ecosystem”. By way of alternative example, user participation in promoting or rating a DTP app (e.g., such as Net Promoter Score), may be captured by way of a survey.

Accordingly, actual patient input, or healthcare provider input, can be used to evaluate the DTP's efficacy, and be used to determine the payor's payment. For example, providers may feel a DTP is great and allows them to avoid needing to prescribe a drug that has side effects and they can enter this as valuable. Alternatively, patients using the app may have fewer side effects and this data, while not necessarily related to the diagnosis are highly values to the patient and payor. The data on side effects can come from questions/a questionnaire within or associated with the DTP, or from claims data. For instance, a drug for depression may increase the risk of falling and decreasing the number of falls in patients with depression because they didn't need the drug and were treated with the DTP is very valuable. This can be gleaned from claims and EMR data. This allows for evaluation of the benefits of the app on a particular patient (which may be used to define success of the DTP app for a particular patient, and to be the basis for determining a success-based payment, which can vary based on the degree of success, to be paid by the payor).

Additionally, DTP ratings by patients, clinicians, etc. may be stored as survey data by the DTP or surveys sent to users of the DTP or drug system, or may be retrieved from the Application Store System 20. In any case, the DTP data is gathered by the DTP Rating Module 160 and associated with a specific DTP. Accordingly, for example, it may be determined that a DTP is highly valued and enjoyed but it may not show clinical improvement. This indicates that the DTP has performed well if this is a metric important to a payor, and the Pricing Module of the TM Engine 150 may take that into account in determining pricing terms, and the Payment Module 180 may implement payment accordingly. Accordingly, actual patient and clinician activities can be used to evaluate the DTP's efficacy, and be used to determine the payor's payment. For instance, in a simple form, a DTP may request at times a “did you like this app?”. Ordinarily such metrics are used to inform the maker of the app about user experience. User experience in health care has been a very challenging domain and rewarding DTP makers for high rates of positive experiencer may be highly valued to stakeholders in the healthcare ecosystem.

Additional patient-specific data may be gathered from an external Platform Data System 70. The Platform Data System 70 is the back-end server/system supporting the corresponding DTP app, such as the backend systems for Pear's reSET-O app or Akili's AKL-T01 app, which are operated or controlled by Pear and Akili, respectively, as shown in FIGS. 1 and 3. Accordingly, data captured at the Platform Data System 70 as a result of the patient's use of the corresponding app may be retrieved from the Platform Data System 70 and be used to advantage by the TM System 100 in determining efficacy of the corresponding DTP. For example, data related to user engagement, use of particular features, or user participation in promoting or rating a DTP app (e.g., such as Net Promoter Score), and such information may be retrieved by the TM System 100 for use in evaluated efficacy/value and determining payment. For instance, high levels of usage, whether this be hourly, daily, weekly, etc. may be something a payor would like to see. For example, the TM System 100 may be configured to gather user engagement metrics as observed by the DTP app.

Additionally, the TM System 100 may gather information from the Patient Computing Device 90, which may be a wearable device or a device on which the user interacts with the DTP. For example, the TM System 100 may communicate with the Patient Computing Device 90 to obtain sleep data captured during the user's use of the DTP via the Patient Computing Device 90. For example, the user may use the DTP app to do CBT and the DTP may record data relating to actual sleep quantity and quality which maybe be indicative of improvement and may be used to advantage in evaluating the DTP's efficacy using the metrics. This allows for evaluation of the benefits of the app on a particular patient (which may be used to define success of the DTP app for a particular patient, and to be the basis for determining a success-based payment, which can vary based on the degree of success, to be paid by the payor). In this manner, the patient's own activities, as reflected in data at the Patient Computing Device 90, can be used to evaluate the DTP's efficacy, and be used to determine the payor's payment. For example, a DTP for insomnia may claim it decreases absenteeism or presenteeism and productivity may be measured via their emails on the device. Or the DTP or drug may claim to improve depression and their mood can be gleaned via NLP from the emails and texts or voice on their calls using the Patient Computing Device 90. For example, the lack of motion of a device, or the lack of user activity on a device, may indicate they are sleeping and this may be linked to a DTP designed to enhance a user's sleep and such data can be used to demonstrate value and success. In another example, a user may have more positive language in their emails and texts and this may demonstrate improved mood and they may have been prescribed a DTP for mood and this may demonstrate improved mood and success without asking them or their clinician directly.

Somewhat similarly, the TM System 100 may interface with other external systems to gather data related to workplace efficacy, absenteeism, presenteeism, etc., so that it can be factored into the evaluation of the DTP app's efficacy, and thus the payor's payment amount for the app. For example, this data may be stored in a communications network-connected HR System (not shown) such as Workday or SAP or disability files (not shown).

### Metric-Based Efficacy Evaluation

Accordingly, as described above, the TM Engine 150 gathers data from various sources that can be used to advantage in accordance with the present invention, using metrics for evaluating DTP efficacy that involve the use of such data.

For example, as discussed above, the TM System 100 may communicate with the Insurance Claims System 30 to obtain medical outcome data gleaned from insurance claims. This may be done by receiving patient record data from the Insurance Claims System 30, or receiving claims record data at the TM System 100 and processing that data to determine outcomes. In either case, the outcome data is received by the Outcome Module 152 and associated with either a specific DTP or a specific patient's outcome after using a specific DTP. Accordingly, for example, it may be determined that discontinuance of claims for a certain condition are indicative of improvement of the condition after using the DTP, such that the patient has achieved a positive outcome from use of the DTP. This indicates that the DTP has performed well, and the TM Engine may take that into account in determining pricing and payment by the payor. For instance, if a DTP or drug is prescribed for a patient, and given to the patient a combination of the following can be known: 1) did the patient use the app (data from the DTP vendor); 2) how did they improve based on clinical scales in the app (such as the PHQ-9); 3) did they have overall lower claims cost from perhaps fewer medical visits; and 4) did they rate their consumer experience with the app as positive and they can be given value based bonuses and payments based on some or all of these. In addition, the system can look at the overall impact on a population of users of the DTP or drug and make special payments based on the positive impact on the entire population. For instance, a base cost for a DTP might be $100/month. But a DTP developer/published may claim that it lowers the cost of care by 50%, it improves sleep by 1 hour per night and that 90% of patients rate the DTP positively. The TM System 100 may be configured to collect data on each of these claims and approve/offer additional payments for the DTP app based on the System's observation of data indicating achievement of such metrics (e.g., payments of $10) for each claimed metric actually achieved when in real-world use.

By way of further example, claims management data gathered from a Claims Management System 40 is received by the Claims Management Module 154 and associated with either a specific DTP or a specific patient's outcome after using a specific DTP. Accordingly, for example, it may be determined that use of the DTP decreased medical visits and the associated costs of those visits. By way of alternative example, a DTP for addiction may have its efficacy evaluated based upon data that a patient had more drug tests that were negative (demonstrating the patient is not using drugs). This indicates that the DTP has performed well, and the TM Engine 150 may take that into account in determining pricing and payment by the payor.

By way of further example, the TM System 100 may communicate with the EMR/EHR System 50 to obtain clinical improvement data gleaned from patient health records. It may also provide options for the patient by combining information on who the payor is and what items are covered for them and based on their EMR language and information may recommend options for the patient. This may be done by receiving patient record data from the EMR/EHR System 50, or receiving claims record data at the TM System 100 and processing that data to determine outcome data. In either case, the health record data is received by the Medical Records Module 156 and associated with either a specific DTP or a specific patient's outcome after using a specific DTP. Accordingly, for example, it may be determined that a patient's weight declined after being prescribed an app for weight loss or for diabetes and the DTP vendor may get rewarded for this. This indicates that the DTP has performed well, and the Pricing Module of the TM Engine 150 may take that into account in determining pricing terms, and the Payment Module 180 may implement payment accordingly.

By way of further example, the TM System 100 may communicate with the Survey System 60 to gather survey data, e.g., via the Survey Module 162, that is associated with either a specific DTP or a specific patient's outcome after using a specific DTP. Accordingly, for example, it may be determined that patients do not enjoy using the DTP and its use is frustrating. A DTP developer/publisher may receive a reduced payment, or no payment, for their DTP as a result. This indicates that the DTP has not performed well, and the Pricing Module of the TM Engine 150 may take that into account in determining pricing terms, and the Payment Module 180 may implement payment accordingly.

The Pricing Module 170 of the TM Engine 150 determines pricing and/or payments for the DTPs or other therapeutic products, based upon therapeutic product efficacy determined by the relevant metrics (namely those elected by the associate payor) in view of the data gathered for evaluation of the efficacy using the metrics, as described above.

The Pricing Module 170 evaluates the efficacy of a particular DTP app, based on the associated metrics elected for a given payor. This is performed using predetermined logic of the Pricing Module 170, and it involves, for example, identifying the relevant metric based on the stored Payor Metric Election for a given DTP, identifying the corresponding Metric Definition 144, and processing the gathered data as associated with the relevant metric definition, to evaluate the efficacy of the DTP based on the metric, in view of the gathered data. The metric and data may involve an assessment of data in relation to a particular patient, or may relate to past or prior efficacy relative to other patients, according to the nature of the metric. The Pricing Module 170 or metric definitions may provide that one or more thresholds must be met for evaluating DTP efficacy. The Pricing Module 170 may be capable of making binary (success/failure or compliance/non-compliance) evaluations of efficacy, or may be capable of assessing a degree of success/efficacy, e.g., on a numerical scale. For instance, a threshold of “20% improvement” on a clinical scale such as blood pressure could be the basis for getting paid or not getting paid for the DTP. Alternatively, payment could be a set amount (e.g., $10) for each increment/number of improvement, such as a change in systolic blood pressure from 140 to 130 is 10 points, and therefore payment would be $100.

By way of further example, the TM System 100 may communicate with the Platform Data System 70 of a particular DTP so that data is gathered by the Platform Data Module 166 and associated with a specific DTP. Accordingly, for example, it may be determined that a questionnaire in the DTP app, such as a PHQ9, shows that a user is feeling better, and this data can be shared with the system and payment may be made as a result. This indicates that the DTP has performed well, and the Pricing Module of the TM Engine 150 may take that into account in determining pricing terms, and the Payment Module 180 may implement payment accordingly. Accordingly, actual patient activities can be used to evaluate the DTP's efficacy, and be used to determine the payor's payment. This allows for evaluation of the benefits of the app on a particular patient (which may be used to define success of the DTP app for a particular patient, and to be the basis for determining a success-based payment, which can vary based on the degree of success, to be paid by the payor).

By way of further example, the TM System 100 may communicate with the patient's Mobile Computing Device 90, in which case data is gathered by the User Data Module 158 and associated with either a specific DTP or a specific patient's outcome after using a specific DTP. Accordingly, for example, it may be determined that a patient is not using the DTP app and therefore engagement is low, or there is no engagement, and therefore there would be no payment for the DTP. This indicates that the DTP has performed well, and the Pricing Module of the TM Engine 150 may take that into account in determining pricing terms, and the Payment Module 180 may implement payment accordingly. Accordingly, an actual patient's activities may be observed, and be used to evaluate the DTP's efficacy, and be used to determine the payor's payment. For example, a payor may want to pay for the level of engagement and usage of the DTP and this kind of app data can be captured and shared. This allows for evaluation of the benefits of the app on a particular patient (which may be used to define success of the DTP app for a particular patient, and to be the basis for determining a success-based payment, which can vary based on the degree of success, to be paid by the payor).

### Efficacy-Based Pricing

Based on the evaluation of the DTP's efficacy as described above, the Pricing Module 170 then determines a corresponding pricing that the payor will, should, or has agreed to, pay for the DTP. The Pricing Module 170 of the TM Engine 150 determines pricing and/or payments for the DTPs or other therapeutic products, based upon therapeutic product efficacy determined by the relevant metrics (namely those elected by the associate payor) in view of the data gathered for evaluation of the efficacy using the metrics, as described above. For example, for a particular payor, or for a particular patient's claim, the Pricing Module 170 may retrieve and reference the applicable metric elected by the payor by retrieving it from the Payor Metric Elections 146 in the Data Store 140, retrieve the associated metric definition from the Metric Definitions 144 in the Data Store 140, and evaluate DTP efficacy, e.g., in a prescribed or predetermined fashion, as a function of the metric and the outcome data received from the Insurance Claims System 30, the Claims Management System 40, the EMR/HER System 50, the Survey System 60, the Platform Data System 70, and/or the Mobile Computing Device 90, depending upon which data is required for evaluating efficacy for the metric elected by the payor. For example, a DTP may have its pricing tied to engagement, 3 metrics of improvement (user experience, clinical scale improvement and total cost of care). Each of these may represent 33% of the total potential price paid for the DTP and each can be measured against the various potential data sources. A payment may be made to a DTP maker in advance and result in a future rebate or payment may be held until a metric is reached, at which point the associated payment may be released. In the case of engagement this may be known immediately, but in the case of cost of care this may not be known for months or even years.

More particularly, the Pricing Module 170 determines, based on predetermined rules of the pricing module and/or of the metric definition, an appropriate payment amount, payment schedule, rebate and/or other payment terms as a result of the evaluation of the efficacy of the DTP, as efficacy is defined by the applicable metric elected by the payor. For example, a payor may want to incentivize more access to care for a condition and may simply want to pay a 10% bonus based on engagement levels with a DTP. They may alternatively (or in addition) want to pay for actual clinical improvement and may pay 20% more for remission of the condition, or 5% for each 10% improvement on a clinical scale. They might also say they will share the financial savings with the DTP vendor. The vendor may feel that the DTP will lower the cost of care by 50% over 5 years and funds can be paid in the future for achieving this metric as compared to historical costs or non-users of the DTP.

This determination of pricing may be performed formulaically by making a mathematical calculation as prescribed by the Pricing Model or a relevant metric, or by reference to logic and/or a pricing structure established by the TM System, the payor, or another, and stored in the Data Store. For instance, it may be decided that payment will be based on a QALY (quality life year) and a dollar amount may be assigned to a QALY such as $50,000. Payment can be made after the success in generating additional QALY for a user are calculated based on the metrics collected. The pricing determined by the Pricing Module may be a payment amount, a series of payments, or involve a rebate or refund, e.g., after subsequent data has been gathered, or may involve a denial of payment, or a requirement of a co-payment by a patient or another. For example, the Pricing Module may be configured to determining pricing involving delaying, sequencing, or holding payments in escrow, based on future patient outcomes, such as death rates. The TM system may also be configured to provide for shared payment for DTPs by the payor and an individual jointly. Optionally, the TM System 100 can set minimum efficacy evaluation thresholds for any payment by payors or individuals. Pricing based on past efficacy could be based on looking at results across selected metrics for the prior period of time, for instance 12 months, and applying this for the subsequent 12 months.

By way of example, data retrieved from the Insurance Claims System and/or Claims Management System 40 may be used to determine a savings in the cost of care are shared with the drug or DTP provider, and the Pricing Module 170 may determine pricing/payments involve the payor's payment of a certain percentage of those savings.

After the payment terms have been determined by the Pricing Module 170, the processing of the associated payment, payments, rebates, etc. to make the payment(s) may be implemented by the Payment Module 180 of the TM Engine 150. This may involve communication of data or transmission of payment instructions to an external payment system (not shown), or to the Payor System 80.

Thus, according to certain embodiments of the present invention, there is provided a method of managing distribution of, and payors' payments for, digital therapeutic products (DTPs) as a function of the various value metrics, and those metrics may account for a particular patient's outcome/result for a particular therapeutic product, or a population of patient's actual outcomes/results for a particular therapeutic product, and the value metrics may be defined and/or chosen/elected by each payor, on a per-payor basis, to align the payor's perception of value with the value actually delivered by the associate therapeutic product, so that the payor's actual payment for the therapeutic product can be aligned with the payor's perception of value.

Accordingly, higher payments to the vendors of the apps may be made by the payors when a greater degree of value to the payor is observed. For example, the TM System 100 may determine value-based payments (based on value-based metrics), such as providing for a higher payment for FDA-approved DTP products (if the payor has assigned higher value to such an attribute by selecting a corresponding metric). By way of alternative example, the payor might select a metric that values highly improvement in the particular user's health (by selecting a corresponding metric). In that case, the TM system may determine value-based payments providing for a higher payment when the patient's health improves, or improves beyond a threshold, etc. These analyses can be done prospectively to establish a marketplace of DTPs, or can be done retrospectively based on a particular use by a patient, or both.

Optionally, the system can also link payment level to whether that patient has a condition for which the application has data associated with success. This would avoid prescribing/paying for a DTP for a patient who is not likely to benefit from it.

Alignment of Payments with Value Delivery

Accordingly, the TM System 100 provides various ways to measure value to payors, by permitting payors to choose (by choosing or even defining efficacy/value metrics) that matter to the payors. Further, the system provides for way to collect efficacy, and thus value, data in real time, using new sources of data, while still interfacing with legacy systems such as claims processing systems.

Additionally, the TM System 100 allows for tailoring of payor payments based on shared/cross-patient data, individual patient outcomes (e.g., successful blood pressure control, depression scale assessment, clean urine for substance abuser, etc.), and group usage data. The TM system may be used to reduce deaths and hospitalizations, and also to reward workplace data, such as absenteeism and presenteeism data. The TM System can also reward consumer experience, such as use of a Net Promoter Score, user ratings of the app, etc.

The TM System identifies appropriate applications to cover, crafts criteria to show payors what the value of each app is, let's payors choose which “value” points are key for them, allow for “value based payments” based on these, creates new ways to pay, hold payments based on outcomes, create incentives for data sharing and product improvement

Some of the features of the TM System include prepopulated criteria around which value can be calculated, links to third party centers such as academic research centers, government databases, etc. to see outcome data in studies, links to actual EMR data with the product impact measures, dashboards to allow payors (plans and employers and the government) to choose the types of products they want, and the “values” that matter to them. For instance, health plan A may be interested in reducing hospital readmissions over 30 days. Plan B may be interested in medication compliance. Plan C may be interested in keeping blood pressure controlled. The dashboard lets them look for products that have proven to do that, see the level of proof and “pre negotiates” payment variable based on the purported value of the product, can hold funds in escrow while long term metric data is collected and analyzed, and combines negotiating power of the multiple payors on the system

### Example

For example, a DTP has been approved called ReSet from Pear Therapeutics. It is not clear what the value generated for this DTP will be to those who would pay for it. The Claims Management System 40 collects past and future payments the payor has and will make. The DTP app on the patient's Mobile computing Device 90, as well as the Platform Data System 70, collects use data, experience data, and other data. The EMR/HER System 50 collects clinic visit data, and lab/blood test/drug test results. All of this data is analyzed by the TM System 100, which can determine a efficacy-based payment structure by processing claims, user, experience data, medical record data, etc. to determine efficacy and price the product accordingly. For instance a payor (insurance company) might want to be able limit costs to no more than $5000 per member (patient) who receives a DTP. The system may be configured to determine 20% of the value according to a Net Promoter Score the DTP achieves, 20% according to the individual's avoidance of a relapse within 24 months, 20% according to observed engagement levels with the DTP, and 40% as a base fee. These values can be altered and adjusted within the TM System 100 via a GUI it provides. In another example, a payor may want to link the payment for a DTP to actual outcomes of a particular patient. For instance, if an insomnia DTP is monitored and tracked within the TM System 100 and the DTP captures actual improvement in sleep, the TM System 100 may assess efficacy and determine pricing/payment according to additional increments of sleep measured and achieved in a direct ratio to the individual's improvement or to the average improvement in a population. The data may be collected in the same device the DTP is loaded on or in another device such as a smart watch or from EMR data.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A therapeutics management system comprising:
a processor;
a memory operatively connected to the processor, said memory storing executable instructions that, when executed by the processor, causes the therapeutics management system to perform a method for efficacy-based management of therapeutic products, the method comprising:
storing in said memory a data store of predefined definitions for a plurality of metrics, each of said plurality of metrics defining a respective measurement of efficacy for use to evaluate efficacy of therapeutic products;
storing in said memory a data store of payors' elections of a least one of said plurality of metrics for use to evaluate at least one therapeutic product; and
for a particular payor and a particular therapeutic product:
retrieving from said memory the particular payor's election of at least one metric;
retrieving from said memory a respective metric definition for each of metric identified by the particular payor's election;
identifying at least one data element required for evaluating efficacy of the particular therapeutic product in accordance with the metric definition;
communicating, via a communications network, with an external computing system to retrieve the at least one data element;
determining efficacy of the particular therapeutic product using the at least one data element in accordance with each respective metric definition; and
determining a prescribed payment to be paid by the payor for the particular therapeutic product as a function of the determined efficacy of the particular therapeutic product.

2. The system of claim 1, wherein said executable instructions provide said system with a therapeutics management engine, and wherein said system comprises a list of registered digital therapeutic products (DTPs) stored in said memory.

3. The system of claim 1, wherein said data store of predefined definitions for a plurality of metrics comprises a definition providing for capture of improvement of a patient on a clinical scale.

4. The system of claim 3, wherein said data store of predefined definitions for a plurality of metrics comprises a definition providing for comparison of the cost of care for patients both with and without use of the DTP.

5. The system of claim 3, wherein said data store of predefined definitions for a plurality of metrics comprises a definition providing for comparison of a rate of hospital readmission for patients both with and without use of the DTP.

6. The system of claim 3, wherein said data store of predefined definitions for a plurality of metrics comprises a definition providing for capture of an indication of the patient's satisfaction when using the DTP.

7. The system of claim 3, wherein said data store of predefined definitions for a plurality of metrics comprises a definition providing for comparison of the patient's current biometric data to previous biometric data.

8. The system of claim 3, wherein said system is configured to cause a graphical user interface to be displayed indicating, for at least one DTP, at least one benefit that the DTP is asserted provide, such that the actual outcome can be compared to the asserted improvement.

9. The system of claim 3, wherein said system is configured to cause display of a graphical user interface providing a dashboard, said dashboard including a display of a list of DTPs, and at least one of a corresponding asserted benefit and a corresponding payor cost for the DTP.

10. The system of claim 9, wherein said system is configured cause a graphical user interface to be displayed, and wherein said dashboard is configured to allow for searching and filtering to allow for display of a subset of the list of DTP that is suitable according to selected criteria.

11. The system of claim 3, wherein said system is configured to cause display of a graphical user interface providing a dashboard, said dashboard including a display of a list of DTPs purporting to provide a benefit for a single diagnosis.

12. The system of claim 11, wherein said dashboard includes a display of data associated with the DTP in use for at least one of academic usage, clinical usage, population usage, patient usage, age-segmented usage, and gender-segmented usage.

13. The system of claim 3, wherein said system is configured to retrieve patient data from an external EMR/EHR, PHR or e-prescribing computing system, and to display the retrieved patient data in a dashboard.

14. A computer-implemented method for efficacy-based management of therapeutic products via a computerized therapeutics management system having at least one processor and a memory operatively coupled to the memory and storing instructions executable by the processor to carry out the method, the method comprising:
storing in the memory a data store of predefined definitions for a plurality of metrics, each of said plurality of metrics defining a respective measurement of efficacy for use to evaluate efficacy of therapeutic products;
storing in the memory a data store of payors' elections of at least one of said plurality of metrics for use to evaluate at least one therapeutic product; and
for a particular payor and a particular therapeutic product:
retrieving from the memory the particular payor's election of at least one metric;
retrieving from the memory a respective metric definition for each of the at least one metric identified by the particular payor's election;
identifying at least one data element required for evaluating efficacy of the particular therapeutic product in accordance with the metric definition;
communicating, via a communications network, with an external computing system to retrieve the at least one data element;
determining efficacy of the particular therapeutic product using the at least one data element in accordance with each respective metric definition; and
determining a prescribed payment to be paid by the payor for the particular therapeutic product as a function of the determined efficacy of the particular therapeutic product.

15. The method of claim 14, further comprising:
causing a graphical user interface to be displayed, the graphical user interface permitting a user to add at least one therapeutic product to a list of registered therapeutic products stored in the memory.

16. The method of claim 14, further comprising:
causing a graphical user interface to be displayed, the graphical user interface displaying at least one of the list of registered therapeutic products, the list of metrics, and the list of metric definitions.

17. The method of claim 14, further comprising:
causing a graphical user interface to be displayed, the graphical user interface displaying metric-based efficacy data indicating efficacy for at least one therapeutic product, as determined by at least one metric.

18. The method of claim 14, further comprising:
causing a graphical user interface to be displayed, the graphical user interface permitting a payor to enter and store an election of at least one metric.

19. The method of claim 14, further comprising:
causing a graphical user interface to be displayed, the graphical user interface permitting a payor to define at least one metric for measuring efficacy for a therapeutic product.

20. The method of claim 14, wherein the external computing system comprises an electronic medical records system, wherein the at least one data element comprises patient outcome data, and wherein determining the prescribed payment is determined as a function of the efficacy of the particular therapeutic product as reflected in the patient outcome data and received electronically at the therapeutics management system.

21. The method of claim 14, wherein the external computing system comprises at least one electronic medical records system, wherein the at least one data element comprises patient outcome data for a plurality of patients, and wherein the prescribed payment is determined as a function of the efficacy of the particular therapeutic product as reflected in aggregated patient outcome data for a population of multiple patients and received electronically at the therapeutics management system.

22. The method of claim 14, wherein the external computing system comprises at least one claims management system, wherein the at least one data element comprises claims data for a plurality of patients, and wherein the prescribed payment is determined as a function of the efficacy of the particular therapeutic product as reflected in claims data for a population of multiple patients and received electronically at the therapeutics management system.

23. The method of claim 14, wherein the external computing system comprises at least one survey system, wherein the at least one data element comprises survey data for a patients, and wherein the prescribed payment is determined as a function of the efficacy of the particular therapeutic product as reflected in the survey data and received electronically at the therapeutics management system.

24. The method of claim 14, wherein the external computing system comprises at least one survey system, wherein the at least one data element comprises survey data for a patients, and wherein the prescribed payment is determined as a function of the efficacy of the particular therapeutic product as reflected in the survey data and received electronically at the therapeutics management system.

25. The method of claim 14, wherein the external computing system comprises at least one platform data system providing backend support to a digital therapeutic product, wherein the at least one data element comprises user activity data selected from a group consisting of user engagement data, feature use data, and user participation data reflecting one of promoting and rating the digital therapeutic product, and wherein the prescribed payment is determined as a function of the efficacy of the digital therapeutic product as reflected in the user activity data and received electronically at the therapeutics management system.

26. The method of claim 14, wherein the external computing system comprises a patient computing device, wherein the at least one data element comprises user activity data selected from a group consisting of data generated by use by operation of a digital therapeutic product at the patient computing device and user behavior data recorded by the patient computing device, and wherein the prescribed payment is determined as a function of the efficacy of the digital therapeutic product as reflected in the data received electronically at the therapeutics management system.

27. The method of claim 14, wherein the therapeutics management system comprises a pricing module, and where in the pricing module determines a prescribed payment to be paid by the payor for the particular therapeutic product as a function of the determined efficacy of the particular therapeutic product using predetermined logic of the pricing module, using a relevant metric based on a payor's metric election, and a corresponding metric definition, by processing associated data to evaluate the efficacy of a digital therapeutic product based on the metric.

29. The method of claim 14, wherein the pricing module determines that predetermined pricing, stored in the memory of the therapeutics management system, is applicable if at least one predetermined quantitative efficacy threshold is met.

30. The method of claim 29, wherein the pricing module determines pricing formulaically.

31. The method of claim 29, wherein the pricing module determines pricing according to prescribed logic and a pricing structure stored in the memory of the therapeutics management system

32. A computer program product for implementing a method for efficacy-based management of therapeutic products, the computer program product comprising a non-transitory computer-readable medium storing executable instructions that, when executed by a processor, cause a therapeutics management engine of a computerized system to perform a method for efficacy-based management of therapeutic products, the method comprising:
storing in the memory a data store of predefined definitions for a plurality of metrics, each of said plurality of metrics defining a respective measurement of efficacy for use to evaluate efficacy of therapeutic products;
storing in the memory a data store of payors' elections of a least one of said plurality of metrics for use to evaluate at least one therapeutic product; and
for a particular payor and a particular therapeutic product:
retrieving from the memory the particular payor's election of at least one metric;
retrieving from the memory a respective metric definition for each of metric identified by the particular payor's election;
identifying at least one data element required for evaluating efficacy of the particular therapeutic product in accordance with the metric definition;
communicating, via a communications network, with an external computing system to retrieve the at least one data element;
determining efficacy of the particular therapeutic product using the at least one data element in accordance with each respective metric definition; and
determining a prescribed payment to be paid by the payor for the particular therapeutic product as a function of the determined efficacy of the particular therapeutic product.
